(19) United States
(12) Patent Application Publication
(10) Pub. No.: US 2006/0264442 A1
(43) Pub. Date: Nov. 23, 2006

(54) Methods for the treatment of ocular and neurodegenerative conditions in a mammal

(75) Inventors: Guadalupe Ruiz, Corona, CA (US); Edwin U. Padillo, Orange, CA (US); Elizabeth WoldeMussie, Laguna Niguel, CA (US)

Correspondence Address: Robert J. Baran (T2-7H);ALLERGAN, INC., Legal Department, 2525 Dupont Drive, Irvine, CA, 92612, US

(73) Assignee: Allergan, Inc.

(21) Appl. No.: 11/133,922

(22) Filed: May 18, 2005

(51) Int. Cl. A61K 31/517

(52) U.S. Cl. 514/252.17; 514/266.2

(57) ABSTRACT

Method for the treatment of conditions including ocular and neurodegenerative conditions in a mammal using a composition which comprises an effective amount of an alpha 1 adrenoreceptor antagonist.

### BACKGROUND OF THE INVENTION

1. Field of the Invention

This invention relates to a method for the treatment of conditions including ocular and neurodegenerative conditions in a mammal using a composition which comprises an effective amount of an alpha 1 adrenoreceptor antagonist. The alpha 1 adrenergic antagonist may be selected from the group consisting of urapidil, prazosin, bunazosin, terazosin, and doxazosin.

2. Description of the Art

Human adrenergic receptors are integral membrane proteins which have been classified into two broad classes, the alpha and the beta adrenergic receptors. Both types mediate the action of the peripheral sympathetic nervous system upon binding of catecholamines, norepinephrine and epinephrine.

Norepinephrine is produced by adrenergic nerve endings, while epinephrine is produced by the adrenal medulla. The binding affinity of adrenergic receptors for these compounds forms one basis of the classification: alpha receptors tend to bind norepinephrine more strongly than epinephrine and much more strongly than the synthetic compound isoproterenol. The preferred binding affinity of these hormones is reversed for the beta receptors. In many tissues, the functional responses, such as smooth muscle contraction, induced by alpha receptor activation are opposed to responses induced by beta receptor binding.

Subsequently, the functional distinction between alpha and beta receptors was further highlighted and refined by the pharmacological characterization of these receptors from various animal and tissue sources. As a result, alpha and beta adrenergic receptors were further subdivided into three alpha 1, three alpha 2, and three beta subtypes.

Functional differences between alpha 1 and alpha 2 receptors have been recognized, and compounds which exhibit selective binding between these two subtypes have been developed. Thus, in WO 92/00073, the selective ability of the R(+) enantiomer of terazosin to selectively bind to adrenergic receptors of the alpha 1 subtype was reported. The alpha 1/alpha 2 selectivity of this compound was disclosed as being significant because agonist stimulation of the alpha 2 receptors was said to inhibit secretion of epinephrine and norepinephrine, while antagonism of the alpha 2 receptor was said to increase secretion of these hormones. Thus, the use of non-selective alpha-adrenergic blockers, such as phenoxybenzamine and phentolamine, was said to be limited by their α₂ adrenergic receptor mediated induction of increased plasma catecholamine concentration and the attendant physiological sequelae (increased heart rate and smooth muscle relaxation or contraction).

Additionally, a method for measuring alpha agonist activity and selectivity comprises the RSAT (Receptor Selection and Amplification Technology) assay as reported in Messier et al., High Throughput Assays Of Cloned Adrenergic, Muscarinic, Neurokinin And Neurotrophin Receptors In Living Mammalian Cells, Pharmacol. Toxicol. 76 (5):308-11 (May 1995) and adapted for use with alpha 2 receptors. The assay measures a receptor-mediated loss of contact inhibition that results in selective proliferation of receptor-containing cells in a mixed population of confluent cells. The increase in cell number is assessed with an appropriate transfected marker gene such as β-galactosidase, the activity of which can be easily measured in a 96-well format. Receptors that activate the G protein, G_{q}, elicit this response. Alpha₂ receptors, which normally couple to Gᵢ, activate the RSAT response when coexpressed with a hybrid Gq protein that has a Gᵢ receptor recognition domain, called G_{q/i5}². See Conklin et al., Substitution Of Three Amino Acids Switches Receptor Specificity Of G_{q}a To That Of Gᵢ alpha, Nature 363 (6426):274-6. (May 20, 1993)

For a general background on the alpha-adrenergic receptors, the reader's attention is directed to Robert R. Ruffolo, Jr., alpha-Adrenoreceptors: Pharmacol Ther. 1994; 61(1-2)1-64, wherein the basis of α₁/α₂ subclassification, the molecular biology, signal transduction, agonist structure-activity relationships, receptor functions, and therapeutic applications for compounds exhibiting α-adrenergic receptor affinity was explored.

The cloning, sequencing and expression of alpha receptor subtypes from human tissue sources has led to the subclassification of the alpha 1 adrenoreceptors into alpha 1A, alpha 1B, and alpha 1D. Similarly, the human alpha 2 adrenoreceptors have also been classified alpha 2A, alpha 2B, and alpha 2C receptors. Each alpha 2 receptor subtype appears to exhibit its own pharmacological and tissue specificities.

Alpha 2 receptor pan-agonists, such as clonidine and dexmedetomidine, have effective analgesic activity, and are currently generally administered directly to the central nervous system, e.g., intrathecally or epidurally for this purpose. Such alpha 2 receptor pan-agonists have sometimes been used for the treatment of chronic pain, such as cancer pain, post-operative pain, neuropathic pain, allodynia, post-herpetic neuralgia, irritable bowel syndrome, and other visceral pain. Other conditions in which such alpha 2 pan-agonists have been shown to have some therapeutic activity include addiction therapy (e.g., opiate or smoking detoxification), attention deficit-hyperactivity disorder (ADHD), Tourette's syndrome, depression and other psychiatric disorders, hypertension, ocular hypertension (such as that associated with some forms of glaucoma), and spasticity. However, these agents have not been commonly and effectively used as analgesic agents or in the treatment of such other indications, due to a very narrow therapeutic window between the therapeutic effect and significant and sometimes overwhelming cardiovascular and sedative activity, as well as a significant interaction with other medications associated with the sedative effects. For examples of the latter, see e.g., Higuchi H., et al., The Interaction Between Propofol And Clonidine For Loss Of Consciousness, Anesth. Analg. 94(4): 886-91, (April 2002); Jaffe, R., et al., Adverse interaction between Clonidine and Verapamil, Ann Pharmacother. 28(7-8):881-3 (July-August 1994)(reporting on the potentially fatal synergism between sedative effects of clonidine and verapamil).

Because of common side effects including high sedative and cardiovascular depression activities at therapeutic doses, FDA approved alpha 2 receptor agonists (which to date have included only alpha 2 receptor pan-agonists) have generally been less useful as systemic agents than as local or topically-applied drugs. Thus, the alpha 2 pan-agonist clonidine has been used for the ophthalmologic treatment of high intraocular pressure (IOP) due to, for example, glaucoma. Since the drug is administered directly in the form of a drop to the eye, many of the usual systemic effects can be minimized. Nevertheless, even topical application of such drugs to the eye (which permits systemic delivery through osmosis into the blood vessels in the eye and through the nasolacrimal duct to the nose), does not eliminate these side effects, and thus therapeutically effective doses are still limited by such effects.

Quinazolinyl-amino derivatives useful as alpha 1 adrenoreceptor blockers are disclosed therapeutic agents for the alpha adrenergic system. See WO 95/25726.

### SUMMARY OF THE INVENTION

In a first embodiment of the present invention, the invention is directed to a method for the treatment of neurodegenerative disorders.

The compound utilized in the method of the present invention is selected from the group consisting of alpha 1 receptor antagonists. Examples of alpha 1 receptor antagonists have been well known in the art for years; however, the method of the invention disclosed herein is believed to be presented for the first time in this patent application.

The epidural administration of clonidine, an alpha 2 pan-agonist, has been reported to effectively provide pain relief and motor block during labor similar to those provided by the local anesthetics bupivacaine-fentanyl. Angelo, Reg. Anaesth. & Pain Med. 25:3 (January-February 2000). However, these effects are accompanied by significantly more sedation and cardiovascular effects, such as hypotension, than was seen using the local anesthetics. Similar results are seen with other alpha 2 pan-agonists such as dexmeditomidine. Alpha 2 agonists, for example, alpha 2 receptor pan-agonists, have been used peripherally or non-peripherally for the treatment of chronic pain, such as cancer pain, post-operative pain, post-herpetic neuralgia, irritable bowel syndrome and other visceral pain, diabetic neuropathy, pain associated with muscle spasticity, complex regional pain syndrome (CRPS), sympathetically maintained pain, headache pain, allodynic pain, inflammatory pain, such as that associated with arthritis, gastrointestinal pain, such as irritable bowel syndrome (IBS) and Crohn's disease, and neuropathic pain. However, in each case, treatment with such a compound is limited by a narrow therapeutic window between analgesia on one hand and oversedation on the other.

Likewise, alpha 2 adrenergic agonists such as apraclonidine (a pan-agonist) and brimonidine have been used in ophthalmic formulations for the treatment of glaucoma and other ocular conditions involving high IOP or reduced uveo-scleral outflow of aqueous humor. While administration by installation of the drug directly into the eye reduces the systemic concentration of the drug, and thus the undesired side effects, some absorption or ingestion of the drug does nevertheless occur via installation. Therefore often the dose of drug necessary to most effectively treat high intraocular pressure is limited by the fact that deleterious side effects may also be seen at such concentrations.

By contrast, the present invention embraces methods for treating a mammal, including a human, having a condition responsive to treatment with an alpha 2 activating agent comprising the administration, to said mammal of an alpha 1 adrenergic receptor antagonist to indirectly activate the alpha 2 adrenergic receptor through endogenous norepinephrine, and thereby avoid degree of sedation or cardiovascular depression than is present following administration of an equivalently effective dose of the A2AA.

An “alpha 2 activating agent” or “A2AA” means an alpha 2 agonist, or other compound whose activity results in a direct or indirect activation of the alpha 2 adrenergic receptor.

Notably, though not exclusively, when used to treat macular degeneration, retinitis pigmentosa, and diabetic retinopathy, the present method provides activity similar to obtained by use of compositions comprising the A2AA as the sole agent. Moreover, such administration of an alpha 1 adrenergic antagonist does not appear to substantially affect the dose-response relationship between the drug concentration and the sedative and hypotensive activity usually seen in compositions comprising the A2AA as the sole therapeutic agent.

Preferred routes of administration for the alpha 1 receptor antagonist may be peripheral or non-peripheral and include oral, intravenous, intrathecal and epidural administration. Other possible means of administration include, without limitation, by intrathecal pump, subcutaneous pump, dermal patch, intravenous injection, subcutaneous injection, intramuscular injection, topical cream or gel, or an oral pill, or a combination of such methods. Drug delivery systems, that may be useful in the method of this invention, are disclosed in U.S. application Ser. No. 11/021,947, filed Dec. 23, 2004; Ser. Nos. 10/837,143 and 10/836,911, both filed on Apr. 30, 2004, and CIPs of the last two applications which were filed on Apr. 29, 2005, all of which are hereby incorporated by reference in their entirety.

In addition to the active ingredient, i.e. the alpha 1 adrenergic receptor antagonist, the compositions utilized in the method of the present invention preferably contain one or more pharmaceutically acceptable carriers consistent with the mode of administration chosen. The phrase “pharmaceutically-acceptable carrier” as used herein means a pharmaceutically-acceptable material, composition or vehicle, such as a liquid or solid filler, diluent, excipient, solvent or encapsulating material. Each carrier must be “acceptable” in the sense of being compatible with the other ingredients of the formulation, with the mode of administration, and not injurious to the patient. Some examples of materials which can serve as pharmaceutically-acceptable carriers include, without limitation: (a) sugars, such as lactose, glucose and sucrose; (b) starches, such as corn starch and potato starch; (c) cellulose, and its derivatives, such as sodium carboxymethyl cellulose, ethyl cellulose and cellulose acetate; (d) powdered tragacanth; (e) malt; (f) gelatin; (g) talc; (h) excipients, such as cocoa butter and suppository waxes; (i) oils, such as peanut oil, cottonseed oil, safflower oil, sesame oil, olive oil, corn oil and soybean oil; (j) glycols, such as propylene glycol; (k) polyols, such as glycerin, sorbitol, mannitol and polyethylene glycol; (l) esters, such as ethyl oleate and ethyl laurate; (m) agar; (n) buffering agents, such as magnesium hydroxide, aluminum hydroxide, boric acid and sodium borate, and phosphate buffers; (O) alginic acid; (p) pyrogen-free water; (q) isotonic saline; (r) Ringer's solution; (s) ethyl alcohol; (t) phosphate buffer solutions; and (u) other non-toxic compatible substances suitable for use in pharmaceutical formulations.

In methods for the treatment of neurodegenerative disorders, the following may be helpful as an aid in the understanding of the invention.

As disclosed in the present invention, alpha 1 receptor antagonists are useful for neuroprotection. For example, conditions which can be treated with the alpha 1 receptor antagonists indicated herein which result in fewer side effects according to the method of the invention include, without limitation, neurodegenerative conditions such as Parkinson's disease, Alzheimer's disease, amyotrophic lateral sclerosis and multiple sclerosis; ischemia such as stroke; epilepsy; and neuropathies such as diabetic and ischemic retinopathy. Additionally, psychiatric conditions such as schizophrenia and bipolar disorder are now thought to involve neurodegeneration to some degree.

Based on the administration of alpha 1 antagonists, the present invention provides therapeutic efficacy in the treatment of neurodegenerative conditions with fewer sedative or cardiovascular side effects than would be seen using the alpha 2 adrenergic agonists.

By reducing or preventing neuronal death, an improvement in pathophysiology or symptoms can be appreciated. As used herein, the term “neuronal death” means destruction of a nerve cell resulting from induction of death in response to an insult or abnormality. Not included in the definition of “neuronal death” is non-pathological neuronal apoptosis, such as that which occurs during embryonic development or in self-renewing tissues containing apoptosis-liable neurons, such as the olfactory epithelium. Therefore, the term neuronal death can include non-olfactory neuroepithelial neuronal damage, such as damage of central nervous system neurons such as brain neurons and neuronal damage within non-apoptosis-liable neurons. As used herein, the term “reducing,” when used in reference to neuronal death means preventing, decreasing or eliminating the induction of death in a nerve cell. Reducing neuronal death by administering an effective amount of an alpha 1 antagonist can be an effective method for treating conditions involving neuronal death or dysfunction with minimized sedative or cardiovascular side effects.

As used herein, the term “neurodegenerative condition” means a disorder characterized by progressive nervous system dysfunction. Neurodegenerative conditions include a heterogeneous group of diseases of the central or peripheral nervous system that have many different etiologies. Such conditions can be, without limitation, hereditary, secondary to toxic or metabolic processes, and can result from infection. Neurodegenerative conditions are progressive conditions that can be age associated or chronic. Such conditions can be characterized by abnormalities of relatively specific regions of the brain or specific populations of neurons. The particular cell groups affected in different neurodegenerative conditions typically determine the clinical phenotype of the condition. In particular, neurodegenerative conditions can be associated with atrophy of a particular affected central or peripheral nervous system structure.

Exemplary neurodegenerative conditions include, but are not limited to, Motor Neuron Disease (ALS), Parkinsonian Syndromes, multiple sclerosis, diffuse cerebral cortical atrophy, Lewy-body dementia, Pick disease, mesolimbocortical dementia, thalamic degeneration, bulbar palsy, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, AIDS related dementia, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, diabetic retinopathy, Alzheimer's disease and ophthalmoplegia. The skilled person understands that these and other mild, moderate or severe neurodegenerative conditions can be treated according to a method of the invention.

In another embodiment of the invention are provided a method for the treatment of an ocular disease comprising the administration of an alpha 1 receptor antagonist. In this embodiment therapeutic efficacy can be obtained with lowered sedative and cardiovascular effects as compared to alpha 2 adrenergic agonists.

Examples of ocular conditions that can be treated using a method of the invention include, but are not limited to, glaucoma, including open angle glaucoma, ocular hypertension, maculopathies and retinal degeneration, such as Non-Exudative Age Related Macular Degeneration (ARMD), Exudative Age Related Macular Degeneration (ARMD), Choroidal Neovascularization, Diabetic Retinopathy, Central Serous Chorioretinopathy, Cystoid Macular Edema, Diabetic Macular Edema, Myopic Retinal Degeneration; inflammatory diseases, such as Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpiginous Choroiditis, Subretinal Fibrosis and Uveitis Syndrome, Vogt-Koyanagi-Harada Syndrome, Punctate Inner Choroidopathy, Acute Posterior Multifocal Placoid Pigment Epitheliopathy, Acute Retinal Pigement Epitheliitis, Acute Macular Neuroretinopathy; vascular and exudative diseases, such as Diabetic retinopathy, Central Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angiitis, Sickle Cell Retinopathy and other Hemoglobinopathies, Angioid Streaks, Familial Exudative Vitreoretinopathy; Eales Disease; traumatic, surgical and environmental disorders, such as Sympathetic Ophthalmia, Uveitic Retinal Disease, Retinal Detachment, Trauma, Retinal Laser, Photodynamic therapy, Photocoagulation, Hypoperfusion During Surgery, Radiation Retinopathy, Bone Marrow Transplant Retinopathy; proliferative disorders, such as Proliferative Vitreal Retinopathy and Epiretinal Membranes; infectious disorders, such as Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associate with HIV Infection, Uveitic Disease Associate with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis; genetic disorders, such as Retinitis Pigmentosa, Systemic Disorders with Accosiated Retinal Dystrophies, Congenital Stationary Night Blindness, Cone Dystrophies, Stargardt's Disease And Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum; retinal injuries, such as Macular Hole, Giant Retinal Tear; retinal tumors, such as Retinal Disease Associated With Tumors, Congenital Hypertrophy Of The RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, and Intraocular Lymphoid Tumors.

Ischemia of the neuroretina and optic nerve can arise during retinal branch vein occlusion, retinal branch artery occlusion, central retinal artery occlusion, central retinal vein occlusion, during intravitreal surgery, in retinal degenerations such as retinitis pigmentosa, and age-related macular degeneration.

The ability of the compositions used in the methods of the present invention, such as the administered alpha 1 adrenergic antagonist, to reduce neuronal death or dysfunction can be assessed by analyzing an observable sign or symptom of nerve cell destruction in the presence and absence of treatment with the compound. Initiation of apoptotic death of neurons can have observable effects on cell function and morphology, as well as observable effects on tissues, organs and animals that contain dysfunctional or apoptotic neurons. Therefore, an indicator of neuronal damage can include observable parameters of molecular changes, such as increased expression of apoptosis-induced genes; cell function changes, such as reduced mitochondrial functions; cell morphological changes, such as cell shrinkage and blebbing; organ and tissue functional and morphological changes, such as the presence of an infarct or other lesion, the severity of which can be assessed by parameters including lesion volume and lesion size; physiological changes in animal models, including functional changes, such as loss of motor function, increased mortality and decreased survival, and behavioral changes, such as onset of dementia or loss of memory.

A reduction in an indicator of neuronal damage can be assessed in a cell, tissue, organ or animal by comparing an indicator of neuronal damage in at least two states of a cell, tissue, organ or animal. Thus, a reduction in an indicator of neuronal damage can be expressed relative to a control condition. A control condition can be, for example, a cell, tissue, organ or animal prior to treatment, in the absence of treatment, in the presence of a different treatment, in a normal animal or another condition determined to be appropriate by one skilled in the art.

In particular embodiments of the invention, a method of the invention is practiced by peripheral administration of the alpha 1 adrenergic antagonist. As used herein, the term “peripheral administration” or “administered peripherally” means introducing an agent into a subject outside of the central nervous system. Peripheral administration encompasses any route of administration other than direct administration to the spine or brain. As such, it is clear that intrathecal and epidural administration as well as cranial injection or implantation, while within the scope of embodiments of the invention, are not within the scope of the terms “peripheral administration” or “administered peripherally.”

Peripheral administration can be local or systemic. Local administration results in significantly more of a pharmaceutical composition being delivered to the site of local administration than to regions distal to the site of administration. Systemic administration results in delivery of a pharmaceutical composition to essentially the entire peripheral nervous system of the subject and can also result in delivery to the central nervous system depending on the properties of the composition.

Routes of peripheral administration useful in the methods of the invention encompass, without limitation, oral administration, topical administration, intraocular administration, intravenous or other injection, and implanted minipumps or other extended release devices or formulations. A pharmaceutical composition useful in the invention can be peripherally administered, for example, orally in any acceptable form such as in a tablet, liquid, capsule, powder, or the like; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation.

Another embodiment of the invention is drawn to methods for the treatment of other conditions for which alpha receptor agonists are known to be effective. These include, without limitation, ocular disorders such as ocular hypertension and glaucoma, neurodegenerative conditions, etc.

In this embodiment, topical delivery of the alpha 1 adrenergic antagonist may be preferred for therapeutic delivery to the eye. Topical ophthalmic formulations are well known in the art.

Ophthalmic pharmaceutical compositions may be prepared by combining a therapeutically effective amount of the alpha adrenergic antagonist as the active ingredient, with conventional ophthalmically acceptable pharmaceutical excipients, and by preparation of unit dosage forms suitable for topical ocular use. The therapeutically efficient amount of the active ingredient is typically between about 0.0001 and about 5% (w/w), preferably about 0.001 to about 1.0% (w/w) in liquid formulations.

For ophthalmic applications, preferably solutions may be prepared using a physiological saline solution as a major vehicle. The pH of such ophthalmic solutions should preferably be maintained between about pH 6.2 and pH 7.8 using an appropriate pharmaceutically acceptable buffer system, (such as, without limitation, a borate, tromethamine or phosphate buffer system). The formulations may also contain conventional, pharmaceutically acceptable preservatives, stabilizers and surfactants.

Preferred preservatives that may be used in the ophthalmic topical methods and compositions of the present invention include, but are not limited to, benzalkonium chloride, other polymeric quaternary ammonium preservatives (such as PHMB and Polyquad®), chlorobutanol, thimerosal, phenylmercuric acetate and phenylmercuric nitrate. A particularly preferred class of preservatives are oxidative preservatives, such as stabilized chlorine dioxide, (e.g., Purite® stabilized chlorine dioxide), stabilized oxyborate, and the like.

Surfactants for use in an ophthalmic formulation may include ionic or non-ionic surfactants such as, without limitation, the Triton (e.g., Triton X-100), Tween® (e.g., polysorbate 40; polysorbate 80), and Pluronic® surfactants.

Likewise, various preferred vehicles may be used in the ophthalmic preparations of the present invention. These vehicles include, but are not limited to, polyvinyl alcohol; Carbopols® (polymers of propionic acid, cross-linked with allyl sucrose); Pemulin®; povidone; poloxamers; cellulosics, such as carboxymethyl cellulose, hydroxypropyl methylcellulose, hydroxyl methylcellulose and the like.

It may be preferable to formulate the ophthalmic formulation as an emulsion. If so, the emulsion may be either an oil-in-water emulsion or a water-in-oil emulsion. The emulsion may contain preservatives, and/or vehicles; however, the emulsion will usually contain at least one surfactant, and will contain an emulsifier. The emulsifier may also be a surfactant. One preferred emulsifier is Pemulin®, which is a cross-linked polyacrylate.

Tonicity adjustors may be added as needed or convenient. They include, but are not limited to, salts, particularly sodium chloride, potassium chloride, mannitol and glycerin, or any other suitable opthalmically acceptable tonicity adjustor.

Various buffers and means for adjusting pH may be used so long as the resulting preparation is ophthalmically acceptable. Accordingly, buffers include acetate buffers, citrate buffers, phosphate buffers and borate buffers. Acids or bases may be used to adjust the pH of these formulations as needed.

In a similar vein, a list of ophthalmically acceptable antioxidants for use in the present invention may include, but is not limited to, sodium metabisulfite, sodium thiosulfate, acetylcysteine, butylated hydroxyanisole and butylated hydroxytoluene.

Other excipient components which may be included in the ophthalmic preparations are chelating agents. The preferred chelating agent is edentate disodium, although other chelating agents may also be used in place or in conjunction with it.

The ingredients can be used in the following amounts:

| INGREDIENT | AMOUNT (% w/w) |
|---|---|
| active ingredient | about 0.001-5 |
| preservative | about 0-0.10 |
| vehicle | about 0-40 |
| tonicity adjustor | about 0-10 |
| buffer | about 0.01-10 |
| pH adjustor | Q.s. pH 4.5-7.5 |
| antioxidant | as needed |
| surfactant | as needed |
| purified water | Q.s. to 100% |

The actual dose of the active compounds of the present invention depends on the specific compound, and on the condition to be treated; the selection of the appropriate dose is well within the knowledge of the skilled artisan.

The ophthalmic formulations of the present invention are conveniently packaged in forms suitable for metered application, such as in containers equipped with a dropper, to facilitate application to the eye. Containers suitable for dropwise application are usually made of suitable inert, non-toxic plastic material, and generally contain between about 0.5 and about 15 ml solution.

Preservative-free solutions are often formulated in non-resealable containers containing up to about ten, preferably up to about five units doses, where a typical unit dose is from one to about 8 drops, preferably one to about 3 drops. The volume of one drop usually is about 20-35 .mu.l.

In specific embodiments, the α1 adrenergic receptor antagonist is selected from the group consisting of prazosin and terazosin and 5-methylurapadil. The former two compounds and their syntheses are described in U.S. Pat. Nos. 3,511,836, and 4,026,894, respectively; the latter compound is an easily synthesized derivative of urapidil, whose synthesis is described in U.S. Pat. No. 3,957,786. See also U.S. Pat. Nos. 5,798,362 and 5,959,108 for synthesizing ∘1 antagonists that are useful in the practice of this invention. These and all other references cited in this patent application are hereby incorporated by reference herein. Additionally, other alpha 1 receptor antagonists are well known in the art; many such compounds have been clinically approved. See also Lagu, “Identification of α_{1A}-adrenoceptor selective antagonists for the treatment of benign prostatic hyperplasia”, Drugs of the Future 2001, 25(8), 757-765 and Forray et al., 8 Exp. Opin. Invest. Drugs 2073 (1999), hereby incorporated by reference herein, which provide examples of numerous alpha 1 antagonists.

Other embodiments will be apparent to one of skill in the art in light of the present specification.

### BRIEF DESCRIPTION OF THE DRAWINGS

FIG. 1 shows the neuroprotective effect of Prazosine, Brimonidine and a combination thereof on retinal ganglion cells.

FIG. 2 shows the effect of varying amounts of Terazosine on retinal ganglion cells.

FIG. 3 shows the effect of 5-methyl-Urapadil on retinal ganglion cells.

FIG. 4 shows the effect of Prazosine for minimizing the elevation of intraocular pressure.

FIG. 5 shows the effect of Prazosine on maintaining retinal function in a model of chronic ocular hypertension.

FIG. 6 shows the preservation of oscillatory potential response by Prazosine in a model of chronic ocular hypertension.

FIG. 7 shows the effect of Prazosine on retinal ganglion cell survival after three months of elevated intraocular pressure.

### DETAILED DESCRIPTION OF THE INVENTION

In a first embodiment, the present invention is related to the Applicants' surprising discovery that peripheral or, alternatively, non-peripheral administration to a mammal in need thereof of a composition comprising: a component comprising an alpha 1 adrenergic receptor antagonist results in indirectly stimulating alpha 2 adrenergic receptor activity. This means that therapeutically effective effects, such as ocular hypotensive activity and neuroprotection may be obtained by administration of an effective amount of an alpha 1 adrenergic antagonist to mammals, including humans and resulting in greatly decreased sedation and cardiovascular depression at a therapeutic dose as compared to administration of a similarly effective therapeutic dose of an alpha 1 adrenergic agonist. In a presently preferred embodiment, the mammal is treated to prevent damage to the optic nerve or retinal neurons.

In this embodiment of the invention, therefore, the Applicants have discovered that the administration of a compound whose activity results in the indirect activation of the alpha 2 adrenergic receptor (A2AA), by an alpha 1 receptor antagonist results in an “unmasking” or enhancement of the therapeutic activity of the endogenous norepinephrine without a substantial increase in the sedative side effects commonly seen upon the use of adrenergic agents which are active at the alpha 1 and alpha 2 receptors.

While the invention is not to be construed as being limited by any particular theory, Applicants believe the invention functions because of blocking of the alpha 1 receptor(s), or one or more of the subtypes thereof, comprising the alpha 1A, alpha 1B and alpha 1D receptor in humans, causes an attenuation of the (e.g., analgesic, ocular hypotensive, neuroprotective, or other) activity resulting from a stimulation of the alpha 2A, alpha 2B, and/or alpha 2C receptor(s). It is also believed that the majority of A2AA's have not been determined to lack alpha 1 stimulatory activity (regardless whether described as being alpha 2 “selective” or not), and therefore possess sufficient intrinsic alpha 1 agonist activity to cause this attentuation effect.

Thus, it is believed that administration of an alpha 1 antagonist blocks the undesired opposing effects caused by stimulation of the alpha 1 receptor. Preferably the alpha 1 antagonist has at least alpha 1A receptor antagonist activity, alpha 1B antagonist activity or alpha 1D receptor antagonist activity. Most preferably, the alpha 1 receptor antagonist has at least alpha 1A receptor antagonist activity. Such antagonist may have antagonist activity at more than one alpha 1 receptor subtype.

The administration to a mammal of an alpha 1 antagonist, in addition to not increasing and sometimes reducing the sedative side effects seen upon treatment of a mammal with an alpha 2 agonist, also improves the therapeutic effect of endogenous norepipinephrine thereby achieving the advantages of administering A2AA without causing the side effects associated with administering an A2AA to a mammal.

In another embodiment, the present invention concerns a method for the treatment of macular degeneration, retinitis pigmentosa, and diabetic retinopathy by administration of a composition comprising an effective amount of an alpha 1 adrenergic receptor antagonist. Preferably, the alpha 1 adrenergic receptor antagonist is selected from the group consisting of urapidil, prazosin, terazosin, bunazosin and doxazosin.

Further, in another embodiment the invention involves methods for the protection of retinal or optic nerve neurons in a mammal comprising administration of a component comprising an alpha 1 adrenergic receptor antagonist. In this embodiment, the more favored route of administration may be a topical ophthalmic formulation, such as a solution, suspension, emulsion, or ointment or a drug delivery system.

Thus, the present invention provides new drug delivery systems, which include an α1 adrenergic receptor antagonist and methods of making and using such systems, for extended or sustained drug release into an eye, for example, to achieve one or more desired therapeutic effects. The drug delivery systems are in the form of implants or implant elements that may be placed in an eye. The present systems and methods advantageously provide for extended release times of α1 adrenergic receptor antagonist. Thus, the patient in whose eye the implant has been placed receives a therapeutic amount of an α1 adrenergic receptor antagonist for a long or extended time period without requiring additional administrations of the α1 adrenergic receptor antagonist. For example, the patient has a substantially consistent level of α1 adrenergic receptor antagonist available for consistent treatment of the eye over a relatively long period of time, for example, on the order of at least about one week, such as between about two and about six months after receiving an implant. Such extended release times facilitate obtaining successful treatment results.

Intraocular implants in accordance with the disclosure herein comprise a α1 adrenergic receptor antagonist and a drug release sustaining component associated there with α1 adrenergic receptor antagonist. The alpha-1 adrenergic receptor agonist may be an antagonist or agent that selectively binds alpha-1 adrenergic receptors. The selective activation can be achieved under different conditions, but preferably, the selective activation is determined under physiological conditions, such as conditions associated with an eye of a human or animal patient. The drug release sustaining component is associated with the therapeutic component to sustain release of an amount of the alpha-2 adrenergic receptor agonist into an eye in which the implant is placed. The amount of the alpha-1 adrenergic receptor agonist is released into the eye for a period of time greater than about one week after the implant is placed in the eye and is effective in preventing or reducing retinal dysfunction. The present intraocular implants are useful prophylaxes that can enhance normal retinal neuronal function. Advantageously, the present intraocular implants may be effective in mitigating against impending retinal neurosensory dysfunction in retinal disorders in patients that may have a predisposition or associated risk factors.

In one embodiment, the intraocular implants comprise an alpha-1 adrenergic receptor agonist and a biodegradable polymer matrix. The alpha-1 adrenergic receptor agonist is associated with a biodegradable polymer matrix that degrades at a rate effective to sustain release of an amount of the agonist from the implant effective to enhance normal retinal neuronal function. The intraocular implant is biodegradable or bioerodible and provides a sustained release of the alpha-1 adrenergic receptor agonist in an eye for extended periods of time, such as for more than one week, for example for about three months or more and up to about six months or more.

The biodegradable polymer component of the foregoing implants may be a mixture of biodegradable polymers, wherein at least one of the biodegradable polymers is a polylactic acid polymer having a molecular weight less than 64 kiloDaltons (kD). Additionally or alternatively, the foregoing implants may comprise a first biodegradable polymer of a polylactic acid, and a different second biodegradable polymer of a polylactic acid. Furthermore, the foregoing implants may comprise a mixture of different biodegradable polymers, each biodegradable polymer having an inherent viscosity in a range of about 0.3 deciliters/gram (dl/g) to about 1.0 dl/g.

The alpha-1 adrenergic receptor agonist of the implants disclosed herein may include quinazolinyl-amino derivatives, or other α1 adrenergic receptor antagonists that are effective in treating ocular conditions. One example of a suitable quinazolinyl-amino derivative is prazosine or prazosine hydrochloride In addition, the present implants may include one or more additional α1 adrenergic receptor and different therapeutic agents other than α1 adrenergic receptor that may be effective in treating an ocular condition.

A method of making the present implants involves combining or mixing the alpha-1 adrenergic receptor antagonist with a biodegradable polymer or polymers. The mixture may then be extruded or compressed to form a single composition. The single composition may then be processed to form individual implants suitable for placement in an eye of a patient.

The implants may be placed in an ocular region to treat a variety of ocular conditions. In addition, the implants are effective in preventing or reducing a symptom of retinal dysfunction, such as by enhancing normal retinal neuronal function. Thus, the present implants may be used as a prophilaxis to promote a healthy retina and reduce symptoms associated with retinal dysfunction.

Kits in accordance with the present invention may comprise one or more of the present implants, and instructions for using the implants. For example, the instructions may explain how to administer the implants to a patient, and types of conditions that may be treated with the implants.

The present invention also encompasses an ophthalmic composition comprising microspheres of an alpha-1 adrenergic receptor antagonist associated with a biodegradable polymer matrix. The microspheres can release the alpha-1 adrenergic receptor agonist upon intravitreal administration or injection of the microspheres.

The ophthalmic composition can upon intravitreal injection enhance normal retinal neuronal function and/or lower intraocular pressure. The alpha-1 antagonist can be a quinazolinyl, such as a diphenylmethyl-1-piperazinyl quinazolinyl-amino, or salts, derivatives thereof and mixtures thereof. For example, the alpha-1 adrenergic agonist can be a prazosine, such as a prazosine freebase or a prazosine salt, such as a prazosine hydrochloride.

The polymer matrix of the ophthalmic composition can comprise a polylactide polyglycolide copolymer. Thus, the ophthalmic composition can comprise microspheres of a prazosine associated with a biodegradable polylactide polyglycolide copolymer, the microspheres being capable of releasing the prazosine upon intravitreal injection of the microspheres.

Our invention also includes a method of making an ophthalmic composition by combining an organic mixture comprising an alpha-1 adrenergic receptor antagonist and a biodegradable polymer, and an aqueous phase, and then stirring the combination to thereby form biodegradable microspheres capable of releasing the alpha-1 adrenergic receptor agonist upon intravitreal injection of the microspheres. The organic phase and the aqueous phase can both be liquids. A detailed embodiment of the method of making an ophthalmic composition can have the steps of: (a) combining an organic mixture comprising a prazosine and a biodegradable polylactide polyglycolide copolymer, and an aqueous phase comprising an aliphatic alcohol, and; (b) stirring the combination to form biodegradable microspheres capable of releasing the prazosin upon intravitreal injection of the microspheres.

Additionally, our invention includes: a method for enhancing normal retinal neuronal function by intravitreal administration of an ophthalmic composition comprising microspheres of an alpha-1 adrenergic receptor agonist associated with a biodegradable polymer matrix, and a method for reducing intraocular pressure comprising the step of intravitreal administration of an ophthalmic composition comprising microspheres of an alpha-1 adrenergic receptor antagonist associated with a biodegradable polymer matrix.

Furthermore, our invention includes a method of treating a retinal dysfunction in an eye of a patient by intravitreal administration of biodegradable intraocular microspheres, the microspheres comprising an alpha-1 adrenergic receptor antagonist associated with a biodegradable polymer matrix that releases alpha-1 adrenergic receptor effective to prevent or reduce a symptom of a retinal dysfunction. The retinal dysfunction treated can be a retinal neurosensory dysfunction and/or an ocular condition such as retinitis pigmentosa, Leber's congenital amaurosis, retinal degeneration, Usher syndrome, Bardet-Biedl syndrome, rod-cone dystrophy, choroideremia, gyrate-atrophy, macular degeneration, and Stargardt's disease.

Finally, our invention encompasses a method for enhancing normal retinal neuronal function and reducing intraocular pressure without significantly obscuring vision by intravitreal administration of an ophthalmic composition comprising microspheres of a prazosine associated with a biodegradable polylactide polyglycolide copolymer polymer matrix.

Materials and Methods

All procedures were conducted following AACUC protocols. Rats were anesthetized by intramuscular administration of Ketamine/Xylazine/Acepromazine/Saline cocktail, 5/2.5/1/1.5, milliliters respectively, at 1 ml/Kg.

Partial Optic Nerve Injury

Sprague Dawley rats weighing 300-350 g were used in this experiment. They were anesthetized with the above anesthetic cocktail and ON injury was applied. A small incision was made on the superior side of the conjunctiva and this was followed by blunt dissection to expose the optic nerve. The tissues around the optic nerve were kept away from the optic nerve using jaw opening forceps. Calibrated forceps was put around the nerve about 3 to 4 mm from the globe and held for 30 seconds. The forceps were then removed, and the eyes treated with topical antibiotics at the site of incision. Drugs were then administered at selected doses via IP. Control animals received vehicle.

Elevation of IOP

Wistar rats weighing 400-475 grams were used. The rats were anesthetized and ocular hypertension was induced by laser photocoagulation of the outflow pathway (mainly episcleral and limbal veins) using a Coherent Argon Laser (Coherent Novus 2000). The number of spots was approximately 150 after two laser treatments with one week interval. IOP was measured using Tono-Pen XL (Mentor O&O). Measurements were done prior to laser treatments to determine baseline and subsequent readings were done weekly.

Ganglion Cell Count

The effect of ON injury and elevated IOP on ganglion cells was determined by evaluating ganglion cell loss in whole mounted retinas. At the end of twelve days for ON injured and three months for COHT animals the ganglion cells were labeled by retrograde transport of Dextran Tetramethyl Rhodamine (DTMR, Molecular Probes, OR). The optic nerve was exposed via surgical incision of the superior conjunctiva followed by retro bulbar blunt dissection. A longitudinal cut (<1 mm) was made at about 3-4 mm distal from the globe. The optic nerve was sectioned and DTMR crystals were applied at the cut end. Twenty four hours after labeling the animals were euthanized and the eyes enucleated and fixed for 2 hours with 4% paraformaldehyde, then the retinas were removed and whole mounted. Cell counts were done using a Nikon microscope under fluorescent light with Rhodamine filter at 400×. Eight to sixteen regions were counted. Ganglion cell loss was calculated as percentage loss compared to unlasered control. Percent protection was calculated by comparing drug treated to vehicle treated eyes. Statistical analysis was done using two-tail student's t-test.

ERG Evaluation

Electroretinographic evaluation of the retina was done only to OHT animals. The retinal function was measured 90 days after first laser treatment and prior retinal labeling. LKC system was used with the following parameters: low and high cut filters 0.3 and 500 Hz respectively, number of runs per sample were 10, flash intensity was −30, −20, −10 and −4 dB, Notch filter was off. Data analysis including OP's extraction from flash ERG was done using the system's software and noise was filtered prior calculation. Data represents the average of 5 animals.

### EXAMPLE 1

### Effect of Alpha1 Antagonists on RGC Protection in ON Injury Model

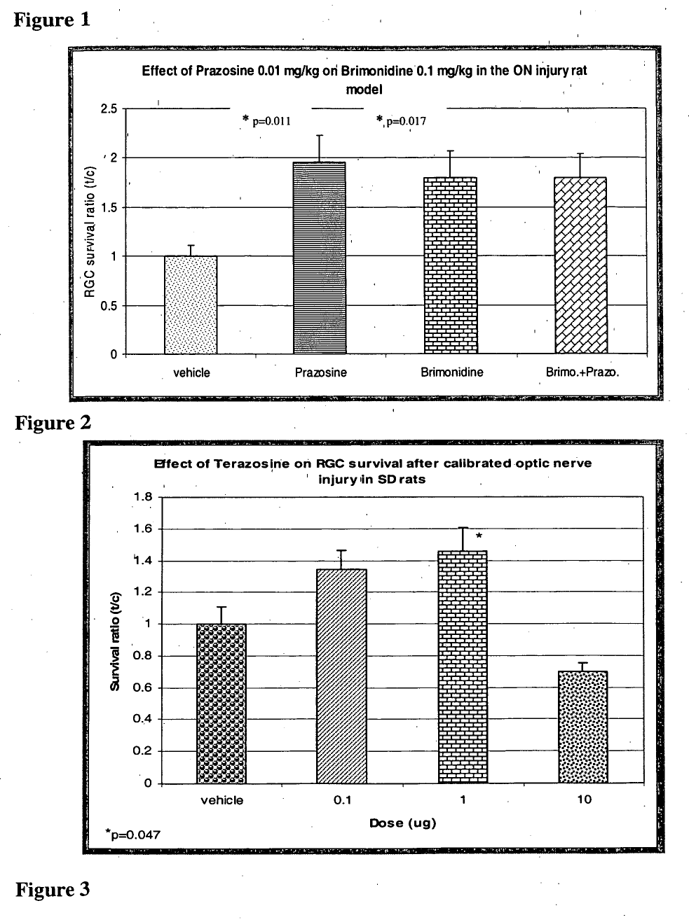

Prazosine at 10 μg/kg resulted in an almost two fold increase in RGC survival. This protective action of Prazosine is similar to that of Brimonidine at 100 μg/kg dose. The co-administration of these two compounds did not show an additive protective effect and the RGC survival was similar to that when the drugs were given individually (FIG. 1). The effect of Terazosine (0.1, 1.0 and 10 μg/kg) is shown in FIG. 2. The results show a biphasic protective effect on RGC. Protection was observed with 0.1 and 1.0 μg/kg with maximum effect with 1.0 μg/kg dose, and the highest dose tested had no effect. The other compound tested, 5-methyl-Urapedil showed no protective effect at 10 μg/kg (FIG. 3). Obviously, this dose was below an effect dose for providing neuroprotection.

### EXAMPLE 2

### Effect of Prazosin on RGC Protection in COHT Model

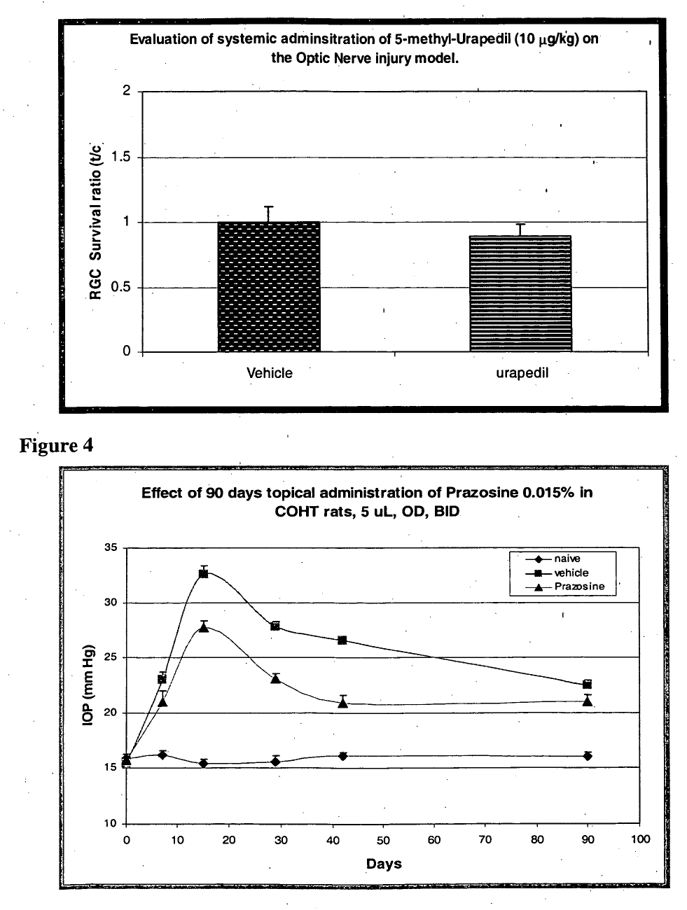

In this model prazosin was tested after topical application of 0.015% twice a day for 90 days. Treatment was initiated after first laser treatment and Prazosine attenuated the elevation of IOP compared to vehicle treated eyes (FIG. 4). The IOP level reached a steady state after 40 days and it was maintained for the rest of the experimental period.

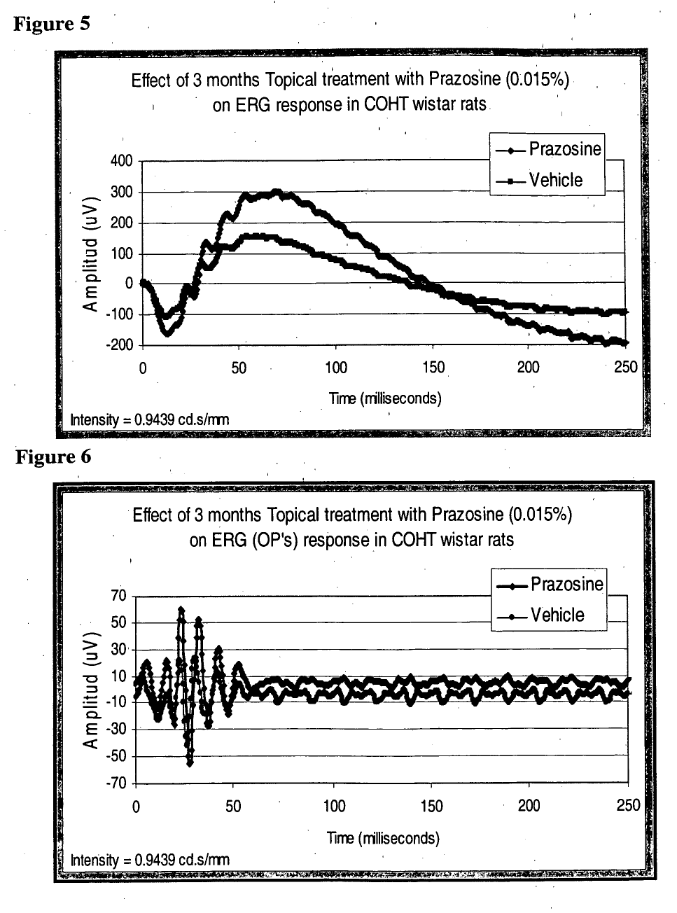

At the end of 90 days retinal function was measured using electroretinography (ERG). In vehicle treated animals both A and B wave amplitudes were smaller than those treated with prazosin (FIG. 5). Similarly the oscillatory potential responses were also preserved by prazosin treatment (FIG. 6).

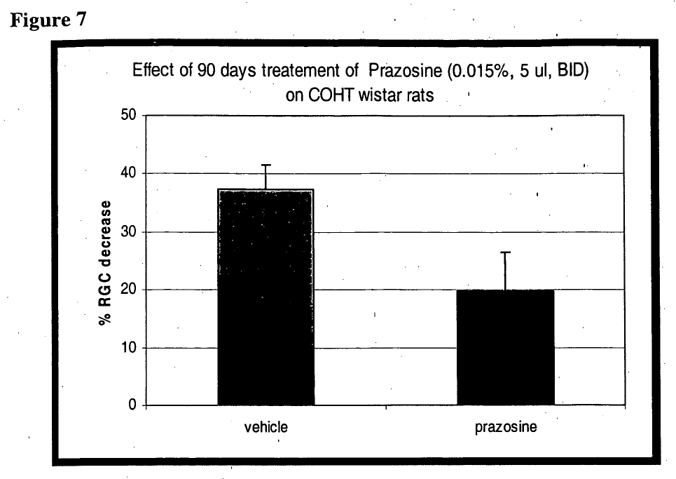

The effect of prazosin on RGC survival is shown in FIG. 7. Three months of IOP elevation resulted in 37% decrease in RGC. In rats that were treated with 0.015% prazosin, the RGC decrease was 20%. This is a 46% protection compared to vehicle treated group. This is comparable to what we have observed with Brimonidine.

Study results have shown that alpha1 adrenergic antagonist compounds are effective in protecting the RGC in the ON injury and COHT rat models; and that Prazosin not only protects the RGC but also has hypotensive activity on IOP and improves the retinal function in ocular hypertensive eyes. These experiments show that blockade of alpha-1 receptors in the eye results in neuroprotection. While not wishing to be bound by theory, it is believed that blocking alpha-1 receptors will provide more of norepinephrine to be available to act on the alpha-2 receptors. Activation of alpha-2 two receptors has been shown to be neuroprotective. Thus the action of Prazosine and other alpha 1 adrenergic antagonists appears to be indirect.

Applicants now present examples for the purpose of illustrating certain embodiment of the invention. The invention is not intended to be limited by these examples.

The following claims are drawn to these and additional embodiments of the invention.

What is claimed is:

1) A method for the treatment or prevention of a neurodegenerative condition in a mammal, comprising the administration of:
a composition comprising an effective amount of an alpha 1 adrenergic receptor antagonist.

2) The method of claim 1 wherein said neurodegenerative condition is selected from the group consisting of Motor Neuron Disease (ALS), Parkinsonian Syndromes, multiple sclerosis, diffuse cerebral cortical atrophy, Lewy-body dementia, Pick disease, mesolimbocortical dementia, thalamic degeneration, bulbar palsy, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, AIDS related dementia, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, diabetic retinopathy, Alzheimer's disease and ophthalmoplegia.

3) The method of claim 2 wherein said alpha 1 adrenergic receptor antagonist is selected from the group consisting of 5-methylurapidil, urapidil, prazosin, bunazosin, terazosin, and doxazosin.

4) The method of claim 3 wherein said alpha adrenergic receptor antagonist is urapidil.

5) The method of claim 3 wherein said alpha adrenergic receptor antagonist is prazosin.

6) The method of claim 3 wherein said alpha adrenergic receptor antagonist is terazosin.

7) The method of claim 3 wherein said alpha adrenergic receptor antagonist is doxazosin.

8) The method of claim 3 wherein said alpha adrenergic receptor antagonist is bunazosin.

9) A method for the treatment or prevention of an ocular condition in a mammal, comprising the administration of:
a composition comprising an effective amount of an alpha 1 adrenergic receptor antagonist.

10) The method of claim 9 wherein said condition is selected from the group consisting of maculopathies, Non-Exudative Age Related Macular Degeneration (ARMD), Exudative Age Related Macular Degeneration (ARMD), Choroidal Neovascularization, Diabetic Retinopathy, Central Serous Chorioretinopathy, Cystoid Macular Edema, Diabetic Macular Edema, Myopic Retinal Degeneration; Acute Multifocal Placoid Pigment Epitheliopathy, Behcet's Disease, Birdshot Retinochoroidopathy, Intermediate Uveitis (Pars Planitis), Multifocal Choroiditis, Multiple Evanescent White Dot Syndrome (MEWDS), Ocular Sarcoidosis, Posterior Scleritis, Serpiginous Choroiditis, Subretinal Fibrosis, Uveitis Syndrome, Vogt-Koyanagi-Harada Syndrome, Punctate Inner Choroidopathy, Acute Posterior Multifocal Placoid Pigment Epitheliopathy, Acute Retinal Pigement Epitheliitis, Acute Macular Neuroretinopathy; Diabetic retinopathy, Central Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angiitis, Sickle Cell Retinopathy and other Hemoglobinopathies, Angioid Streaks, Familial Exudative Vitreoretinopathy; Eales Disease; Sympathetic Ophthalmia, Uveitic Retinal Disease, Retinal Detachment, Trauma, Retinal Laser, Photodynamic therapy, Photocoagulation, Hypoperfusion During Surgery, Radiation Retinopathy, Bone Marrow Transplant Retinopathy; Proliferative Vitreal Retinopathy, Epiretinal Membranes; Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associate with HIV Infection, Uveitic Disease Associate with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis; Retinitis Pigmentosa, Systemic Disorders with Accosiated Retinal Dystrophies, Congenital Stationary Night Blindness, Cone Dystrophies, Stargardt's Disease And Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum; Macular Hole, Giant Retinal Tear; Retinal Disease Associated With Tumors, Congenital Hypertrophy Of The RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, and Intraocular Lymphoid Tumors.

11) The method of claim 10 wherein said alpha 1 adrenergic receptor antagonist is selected from the group consisting of urapidil, prazosin, terazosin, bunazosin and doxazosin.

12) The method of claim 11 wherein said alpha adrenergic receptor antagonist is urapidil.

13) The method of claim 11 wherein said alpha adrenergic receptor antagonist is prazosin.

14) The method of claim 11 wherein said alpha adrenergic receptor antagonist is terazosin.

15) The method of claim 11 wherein said alpha adrenergic receptor antagonist is doxazosin.

16) The method of claim 11 wherein said alpha adrenergic receptor antagonist is bunazosin.

17) The method of claim 11 in which the ocular condition is selected from the group consisting of macular degeneration, retinitis pigmentosa, and diabetic retinopathy.

18) The method of claim 11 in which the ocular condition is macular degeneration.

19) The method of claim 11 in which the ocular condition is retinitis pigmentosa.

20) The method of claim 11 in which the ocular condition is diabetic retinopathy.
